(19) United States
(12) Patent Application Publication
(10) Pub. No.: US 2022/0030913 A1
(43) Pub. Date: Feb. 3, 2022

(54) SUPPLEMENT FOR LIVESTOCK

(71) Applicants: Foote Scott Robert, Hoxie, KS (US); Kreikemeier Kelly, Hoxie, KS (US)

(72) Inventors: Foote Scott Robert, Hoxie, KS (US); Kreikemeier Kelly, Hoxie, KS (US)

(73) Assignee: Hoxie Feedyard, LLC

(21) Appl. No.: 17/393,254

(22) Filed: Aug. 3, 2021

Related U.S. Application Data
(60) Provisional application No. 63/060,513, filed on Aug. 3, 2020.

(51) Int. Cl. A23K 20/132; A23K 20/121; A23K 10/30; A23K 50/10; A23K 20/174; A23K 20/20

(52) U.S. Cl. CPC A23K 20/132; A23K 20/121; A23K 20/20; A23K 50/10; A23K 20/174; A23K 10/30

(57) ABSTRACT

A product and method of parasitic control, ammonia control, methane inhibition, liver abscess inhibition, and nutritional supplementation in livestock in order to grow healthier and more efficient livestock. The supplement is designed to provide an antibiotic-free and antiparasitic-free feed additive to decrease parasites, inhibit methane production, and ammonia production in livestock, inhibit the incidence of liver abscesses while providing a nutritional supplement to livestock and simultaneously providing for increased weight gain and overall positive health in the same.

### FIELD OF INVENTION

The present invention is related to a product and method of parasitic control, reduced methane production, ammonia control, liver abscess inhibition, and nutritional supplementation in livestock. More specifically, the present invention relates to products and methods for antibiotic-free and antiparasitic-free parasite control in livestock, reducing the production of methane from livestock, preventing the volatilization of ammonia from livestock, and providing a nutritional supplement to livestock that leads to optimal weight gain.

### BACKGROUND OF THE INVENTION

A significant problem facing the bovine and livestock industries is illness due to parasitic infection. This issue results in lower production, loss of yield, longer time-to-market, and may cause illness and death to the animal. Simultaneously, producers are constantly seeking products and methods for increasing both the amount of muscle gained and the rate of that gain to satisfy the ever-increasing worldwide demand for livestock products. Bearing those issues in mind, there is a need to evaluate the use of feed grade antimicrobials used in cattle feeding.

Coccidiosis is a parasitic disease of the intestinal tract of animals caused by coccidian protozoa. Coccidia are protozoan parasites that are host-specific (e.g., cattle have their specific coccidia, poultry have their coccidia, etc.). Eimeria zuernii is a species of the parasite Eimeria that causes diarrheic disease known as eimeriosis or coccidiosis in cattle. Both symptomatic animals and asymptomatic carrier animals shed oocytes in their feces promoting spread of the disease. The oocysts mature in moist, warm environments and become infective. Coccidia oocysts are ingested by livestock when they consume contaminated feed, water, forage or simply lick the coat of an infected animal. When ingested, the parasite can develop inside the host animal, causing damage to intestinal cells and lining, resulting in the host animal exhibiting a loss of appetite, fatigue, dehydration, and watery, sometimes bloody, diarrhea. Damage to the intestinal mucosa further impacts the animal's ability to absorb fluids to compensate for the water losses in the diarrhea. The oocyst is highly resilient and can survive in moist, shaded areas for years.

Numerous animals exposed to and infected with coccidia fail to develop symptoms and go on to develop species-specific immunity. Disease generally occurs when large numbers of the infective form of the protozoa (oocysts) are ingested, the animals are too young to have developed an immunity, are stressed, or the animals' immune response is compromised. Weaning, shipping or moving cattle may cause sufficient stress to cause illness and visible symptoms. Sick animals usually have acute diarrhea with or without blood, decreased appetite and mild lethargy. In more severe cases, this may progress to severe lethargy, dehydration, pale mucous membranes, straining and severe weight loss. Some cattle with coccidiosis may present with neurologic symptoms and death may occur despite therapy. The disease commonly affects young animals managed as groups in unsanitary conditions. Calves as young as 16 days of age may be affected. Older cattle are less susceptible to disease than younger cattle unless they are experiencing extreme stress or have compromised immune systems.

Coccidiosis results in reduced growth rates, as well as acting as a stressor, causing increased susceptibility to other infections, such as salmonellosis, or Bovine Respiratory Disease. Coccidiosis is primarily a disease that affects young animals but can affect older animals that are immune compromised. It occurs commonly in confined conditions but can occur in free-ranging conditions that have congregating areas, such as feeding, shade and watering areas.

Coccidiosis causes substantial economic losses due to reduced performance, death from direct infections, and by predisposing livestock to secondary bacterial and viral infections. The labor demand for the treatment and care of infected cattle in addition to medication costs amplify the economic losses.

Methane emission from ruminant livestock is currently estimated to be around 100 million tons each year and, after fossil fuel-related emissions, represents the biggest man-made methane source. The loss of methane from ruminant livestock is a problem not only in the respect of greenhouse gas emissions, but also to farmers in that feed converted into and released as methane is feed not being converted into meat and/or milk. Methane is produced in the gut of ruminant livestock as a result of methanogenic microorganisms. The composition of the animal feed is a crucial factor in controlling the amounts of methane produced, but a sheep can produce about 30 liters of methane each day while large ruminant livestock can produce between 250 and 500 liters of methane per day. Many factors influence methane emissions from cattle and include the level of feed intake, type of carbohydrate in the diet, feed processing, addition of lipids or ionophores to the diet, and alterations in the ruminal microflora. Manipulation of these factors can reduce methane emissions from cattle and cattle farmers are continually searching for products which reduce methane production in the rumen, thereby gaining greater energetic efficiency of the digested feed.

Volatized ammonia is a major problem for farmers of livestock, especially poultry. Agriculture is the major source of ammonia emission to the atmosphere. Studies have confirmed the increased concentration of ammonia in areas of animal agriculture. Studies also indicate that animal agriculture accounts for more than 80% of global ammonia volatization. Ammonia (NH3) is a natural byproduct of chemical reactions in the gut of livestock and from microbial breakdown of manure in bedding litter. Ammonia is present at alkaline conditions (pH greater than 7) and creates a noxious odor. When volatized in the air, ammonia has a negative impact on animal health by harming the respiratory track and eyes. Too much ammonia has also been shown to reduce weight gain in livestock. One strategic mechanism to combat ammonia is to acidify the gut of livestock and the bedding. Under acidic conditions (pH less than 7), ammonia is converted to ammonium (NH4+). Ammonium is not easily volatized into the air, thus having less negative impact on air quality and respiratory health.

Among the largest uses of antimicrobials in finishing cattle are the control of liver abscesses, improved performance, and the prevention of coccidiosis. Liver abscesses are the primary liver disease in feedlot cattle found at slaughter. In the feedyard, liver abscesses result secondary to acidosis and rumenitis, often from high concentrate feeds and aggressive feeding practices. Estimates of liver abnormalities have ranged from 0-70%, and there have been several reports on the economic and production losses associated with liver abscesses. Liver abscesses are most common in feedlot and dairy cattle which are fed rations that predispose to rumenitis. It is most observed in feedlot cattle and in dairy cattle that are fed high level grain diets. Cattle with liver abscesses have reduced production efficiency. Affected livers are condemned at slaughter, and adhesions to surrounding organs or the diaphragm may necessitate carcass trimming. Liver abscesses can also lead to disease syndromes associated with posterior vena caval thrombosis. It is believed that most liver abscesses are associated with episodes of rumen acidosis caused by carbohydrate overload. Risk factors include the amount of carbohydrate ingested, the type of grain being fed and the degree to which the grain has been processed.

Tylosin, a macrolide antimicrobial (Tylan®, Elanco Animal Health) has been used successfully for the reduction of liver abscesses in feedlot diets. Furthermore, ionophores such as monensin (Rumensin, Elanco Animal Health) have been widely for increased feed efficiency, intake regulation, and the control of coccidiosis. While ionophores are not currently on the World Health Organization Critically Important Antimicrobials for Human Medicine list, nor is the use of monensin alone affected by any Veterinary Feed Directive, as consumer preferences continue to change, there is a need to evaluate alternatives to antimicrobials.

Thus, there is clearly a need for both a product and method which is an alternative to antimicrobials, alleviates parasitic affliction, reduces methane production in the gut, and lowers ammonia generation as described above.

### SUMMARY OF THE INVENTION

A product and method which is an alternative to antimicrobials and provides parasitic control, ammonia control, methane inhibition, liver abscess inhibition, and nutritional supplementation in livestock in order to grow healthier and more efficient livestock. The supplement is designed to provide an antibiotic-free and antiparasitic-free feed additive to decrease parasites, inhibit methane production, and ammonia production in livestock, inhibit the incidence of liver abscesses while providing a nutritional supplement to livestock and simultaneously providing for increased weight gain and overall positive health in the same.

### DETAILED DESCRIPTION

The present invention now will be described more fully hereinafter in the following detailed description of the invention, in which some, but not all embodiments of the invention are described. Indeed, this invention may be embodied in many different forms and should not be construed as limited to the embodiments set forth herein; rather, these embodiments are provided so that this disclosure will satisfy applicable legal requirements.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

A multitude of microorganisms inhabit the intestinal tract of ruminants and include bacteria, fungi, and protozoa. Pathogenic microorganisms are shed in the manure of livestock facilitating the passage these bacteria to other livestock. As stated above, protozoa are eukaryotes, one celled animals that are found in many parts of the ecosystem including soil, ponds, and the digestive tract of ruminants. Protozoa consume bacteria as food. Numerous pathogenic bacteria can survive intracellularly in soil protozoa. Even as these soil protozoa become encysted in response to poor environmental conditions, the intracellular pathogens remain viable.

Free-living protozoa are believed to be able to act as a delivery vehicle and carry pathogenic endosymbionts. In this manner, the protozoa can act as a life-preserving capsule for the pathogen. Studies suggest that some Salmonella, when ingested by rumen protozoa, developed the ability to resist the digestive processes of the protozoa and remain viable. Further, reports indicate that the pathogen can become more virulent after living within the protozoa in comparison to pathogens that have not lived inside the protozoa. Rumen microorganisms are swept from the fore-stomach of the ruminant animal into the lower digestive tract. In the lower digestive tract, microorganisms are digested and provide nutrients for the host ruminant. Protozoa are moved more slowly from the rumen of the animal due to their ability to cling to the larger undigested feed and to the rumen walls. Their turnover rate is much slower, and this allows the intracellular pathogens to sustain themselves in the rumen. The rumen then becomes a reservoir for the pathogens. As some of the protozoa are swept into the lower digestive system of the ruminant, the protozoa are digested, releasing the more virulent pathogens. The pathogens that have developed a resistance to the digestive processes survive and are shed in the manure of the host animal.

The instant invention discloses a product and method to combat the production of ammonia, methane and parasites thereby promoting efficient growth of healthier poultry and livestock. Simultaneously, the instant invention promotes increased muscle weight gain and overall positive health in the same. The supplement is designed to reduce ammonia production from animal manure while conditioning the gut of animals to reduce bacterial load and infection without the need for antibiotics. Additionally, the supplement reduces liver abscesses which are seen in all ages and breeds of cattle wherever cattle are raised.

The instant invention involves using a combination of decoquinate and sarsaponin to lessen or eliminate problems caused by parasites, ammonia and excess methane production to act as a nutritional supplementation in livestock. As a system and method to reduce the use of antimicrobials, the instant invention either removes or substitutes tylosin phosphate and monensin from finishing rations for the last 120 days, 90 days, or 60 days without significant changes in cattle performance, liver abscess incidence, or carcass quality at slaughter.

The instant invention includes a food supplement composition for a non-human animal comprising decoquinate and sarsaponin. The decoquinate can be commercially obtained from Zoetis under the tradename DECCOX®. (DECCOX is a registered trademark owned by Zoetis). Decoquinate is a non-antibiotic feed additive for the prevention of coccidiosis in beef cattle and chickens caused by various Eimeria species. The data collected for the instant invention indicates that feeding decoquinate improved cattle growth performance. Additionally, our data goes on to demonstrate that the improved growth performance in cattle fed decoquinate is similar to that of cattle fed monensin and tylosin which are “the gold standard” of feed grade antibiotics fed to finishing cattle today. Although decoquinate is approved as a coccidiostat, we believe that part of the improved growth performance with feeding decoquinate can be attributed to altered rum inal fermentation, which aids weight gain optimization of the livestock as well as decreasing the production of ammonia.

Saponins are a class of chemical compounds found in various plant species. Saponins are natural plant surfactants that occur in over five hundred different plant species belonging to some eighty different families. They are generally recognized by their strong foaming action when placed in water, which has made them especially useful in the manufacture of foods, beverages, shampoos, wetting agents and pharmaceuticals. Saponins are classified as surfactants because they have both lipophilic and hydrophilic “regions”. Thus, the surfactant activity of saponins is a result of both fat-soluble and water-soluble moieties in the same molecule. The lipophilic region may be a steroid, triterpene, or alkaloid, and is termed a sapogenin. The hydrophilic “region” contains one or more water-soluble carbohydrate side chains. The structural complexity of saponins is derived largely from the carbohydrate portion of the molecule due to the many different types of possible side chain carbohydrates, such as glucose, xylose, galactose, pentose or methylpentose, which may have different connectivity and/or anomeric configuration. Saponins have an anti-protozoal activity attributed to the saponin's ability to interact with cholesterol in protozoal cell membranes and cause cell lysis. Types of saponins include steroidal and triterpenoidal.

The instant application makes use of steroidal saponins, (e.g., sarsaponin). The sarsaponin can be obtained from a variety of sources including, but not limited to, sarsaponin extracted from plants of the family: Liliaceae, genus: Yucca, such as Yucca schidigera. Yucca derived saponins generally have steroidal sapogenins. Sarsasapogenin is the major sapogenin found in the Yucca schidigera plant. Saponins useful in the present invention can also be extracted from plants of the family: Amaryllidaceae, genus: Agave, which grows extensively in the southwestern United States and in Mexico. Additional sources of saponins can include extracts of soybeans, fenugreek, peas, tea, yams, sugar beets, alfalfa, asparagus, aloe, vanilla, zhimu, Sapindus saponaria, citrus fruits (Iimonoid saponins) as well as from Quillaja saponaria bark. Saponins can be extracted from plant materials in accordance with techniques well-known by those of skill in the art.

Compositions in accordance with embodiments of the invention can include various other additives. By way of example, compositions can also include components such as, water, propylene glycol, Vitamin E (as di-alpha-tocopheryl acetate), Vitamin A Propionate, Vitamin A Palmitate, Vitamin B1, Vitamin B2, Vitamin B6, Vitamin B12, D-Activated Animal Sterol (source of Vitamin D3), yeast components, dried egg solids, dried casein, and dried whey.

The typical saponin content that naturally occurs in Yucca plants is from 0.1-2% saponins by weight. Yucca extracts can be derived by extracting yucca powder with an aqueous solution that may or may not contain some fraction of organic solvent such as methanol, ethanol, propanol, butanol, or the like.

Commercially available Yucca extracts can have a total solids content usually in the range from 5-50%. The saponin content of a typical 50 brix (50% solids by weight) yucca extract is usually in the range of about 1-2% saponins by weight as measured by HPLC analysis. Another method of measuring total saponin content is the extraction of all soluble components into a butanol extract followed by gravimetric analysis of the compounds dissolved in the butanol fraction. Measuring saponin content by the butanol extract method typically results in higher numbers than the more advanced HPLC method. Accordingly, the typical 50 brix (50% solids by weight) yucca extract is usually in the range of about 5-20.0% saponins content by weight as measured by the butanol extract method.

Saponin containing compositions can also be formulated as dry powder. Such dry formulations are available commercially from numerous suppliers. On a dry matter basis, these dry formulations typically contain up to 30 percent yucca extract solids. Dry powder formulations of saponin containing compositions may be added to the feed ration via a micro-ingredient machine or hand added to a feed mix truck and mixed thoroughly to assure even distribution in the feed. By way of example, a dry formulation described previously can be added at a rate of 0.25 gram to 10 grams per head per day.

Saponin containing compositions in accordance with the invention may be in liquid or dry forms. For example, compositions herein can be formulated as a liquid, slurry, dry powder, dry granular mix, paste, pellets, block, or the like. A saponin containing plant extract may be dried into a powder form. In this form, the saponin containing composition may be administered to an animal as a pill, bolus, or mixed in with other components such as a feed ration. Saponin containing plant extract may also be in a solution with an amount of a carrier liquid such as water. In this form, the saponin containing composition may be administered to an animal as a liquid drench.

Saponin containing compositions may be administered to an animal as a single dose. Saponin containing compositions may also be administered to an animal in multiple doses. For example, an animal may receive an initial dose and then receive subsequent maintenance doses in lesser amounts. An animal may receive multiple doses of a saponin containing composition in one day or may receive multiple doses over multiple days.

Animals can be treated with a saponin containing composition in an amount that is effective to improve the feeding characteristics of an animal and/or improve the production characteristics of an animal in comparison to an untreated control animal. Production characteristics can include carcass quality grades, yield grades, average daily gain, milk production, and the like. In an embodiment, the amount of saponins in a dose of a saponin containing composition is at least about 5 milligrams. In an embodiment, the amount of saponins in a dose of a saponin containing composition is less than about 10 grams. In an embodiment, the amount of saponins in a dose of a saponin containing composition is about 5 mg to 10 grams. The amount of decoquinate in a dose of a decoquinate containing composition is at least about 5 milligrams. In an embodiment, the amount of decoquinate in a dose of a decoquinate containing composition is less than about 10 grams. In an embodiment, the amount of decoquinate in a dose of a decoquinate containing composition is about 5 mg to 10 grams.

Yucca schidigera extract has been fed to various livestock for years. It reduces ammonia emission from the manure in confinement raised chickens and pigs and it has also been shown to exhibit “anti-protozoal” activity in cattle. The addition of Yucca schidigera extract to finishing cattle diets which were also supplied medicated feed additives (i.e., monensin and tylosin) has shown little to no effect on growth performance or weight gain optimization. Surprisingly, for cattle finishing studies completed for the instant invention, cattle fed Yucca schidigera extract in the absence of a diet containing no medicated feed additives, improvements in growth performance were demonstrated which are likely attributed in part to a decrease in methane production.

The instant invention demonstrates that adding decoquinate to a cattle finishing diet not only helps control coccidiosis (mentioned above), but also improves cattle growth performance at levels approaching that of cattle fed antibiotic feed additives (monensin plus tylosin). Previous research has shown that cattle fed decoquinate have improved growth performance. Our research extends that conclusion that the improved growth performance with a combination of both decoquinate and Yucca schidigera extract approaches the levels of rations containing the antibiotic additives monensin plus tylosin.

Cattle fed rations with added decoquinate demonstrate the above when given at the following doses: 0.1 to 5.0 mg/kg body weight (BW); 0.2 to 5.0 mg/kg BW; 0.3 to 5.0 mg/kg BW; 0.4 to 5.0 mg/kg BW; 0.5 to 5.0 mg/kg BW; 0.5 to 4.5 mg/kg BW; 0.5 to 4.0 mg/kg BW; 0.5 to 3.5 mg/kg BW; 0.5 to 3.0 mg/kg BW; 0.5 to 2.5 mg/kg BW; 0.5 to 2.0 mg/kg BW; 0.5 to 1.5 mg/kg BW; 0.5 to 1.0 mg/kg BW; 0.3 to 1.0 mg/kg BW; 0.3 to 0.8 mg/kg BW; 0.3 to 0.5 mg/kg BW; 0.1 to 10.0 mg/kg BW; 0.2 to 10.0 mg/kg BW; 0.3 to 10.0 mg/kg BW; 0.4 to 10.0 mg/kg BW; 0.5 to 10.0 mg/kg BW; 4.5 to 10.0 mg/kg BW; 4.0 to 10.0 mg/kg BW; 3.5 to 10.0 mg/kg BW; 3.0 to 10.0 mg/kg BW; 2.5 to 10.0 mg/kg BW; 2.0 to 10.0 mg/kg BW; 1.5 to 10.0 mg/kg BW; 1.0 to 10.0 mg/kg BW.

Cattle fed rations with added sarsaponin from Yucca Schidigera have improved growth performance and a lower incidence of liver abscesses compared to cattle fed antibiotic free diets. Cattle fed rations with added sarsaponin from Yucca Schidigera demonstrate a reduction in methane production when given at the following doses: 50 to 500 mg/kg feed; 75 to 500 mg/kg feed; 100 to 500 mg/kg feed; 125 to 500 mg/kg feed; 150 to 500 mg/kg feed; 175 to 500 mg/kg feed; 200 to 500 mg/kg feed; 250 to 500 mg/kg feed; 300 to 500 mg/kg feed; 100 to 400 mg/kg feed; 100 to 300 mg/kg feed; 50 to 1000 mg/kg feed; 75 to 1000 mg/kg feed; 100 to 1000 mg/kg feed; 125 to 1000 mg/kg feed; 150 to 1000 mg/kg feed; 175 to 1000 mg/kg feed; 200 to 1000 mg/kg feed; 250 to 1000 mg/kg feed; 300 to 1000 mg/kg feed; 400 to 1000 mg/kg feed; 500 to 1000 mg/kg feed; 600 to 1000 mg/kg feed; 700 to 1000 mg/kg feed; 800 to 1000 mg/kg feed; 900 to 1000 mg/kg feed. The above doses are calculated based on the product available from MICRO-AID GREEN® which contains approximately 30% yucca extract solids (i.e., the first dosage above of 50 to 500 mg/kg feed corresponds to 15 to 150 mg of dried yucca solids and the last dosage above of 900 to 1000 mg/kg feed corresponds to 270 to 300 mg of dried yucca solids).

The combination of decoquinate and sarsaponin in the same ration in the absence of additional antimicrobials results in an unexpected synergistic effect compared to feeding either one separately. The parasitic control, ammonia control, methane inhibition, improved growth and lowered incidence of liver abscesses equal those of rations containing antibiotic feed additives. The ratio of decoquinate to sarsaponin can range from: 1 to 1; 1 to 2; 1 to 3; 1 to 4; 1 to 5; 1 to 6; 1 to 7; 1 to 8; 1 to 9; 1 to 10.

The food supplement composition of the instant invention includes components that may be in liquid form, solid form, or a combination thereof. The physical form is optimally a pellet, a crumble, a mash, or a lick.

The food supplement composition of the instant invention can further include: at least one grain by-product selected from the group comprising oats, barley, maize, lupins, lupin hulls, bran, canola meal, and soya meal; hay, wherein the hay is at least one of oaten, wheaten, and meadow hay; at least one pharmaceutical composition; at least one nutrient, wherein at least one nutrient is at least one of a vitamin and a mineral; a flavoring, such as molasses; a pharmaceutical composition comprising an animal food supplement composition as an active ingredient optionally admixed with a pharmaceutically acceptable carrier; one or more strains of bacteria; one or more essential oils; Yeast; live, dead, cell components, or its fermentation extract; Mold; live, dead, cell components, or its fermentation extract; Lactic acid producing bacteria (LAB); live, dead, cell components, or its fermentation extract; Lactic acid utilizing bacteria; live, dead, cell components, or its fermentation extract; Bacillus bacteria; live, dead, cell components or its fermentation extract; Nutrient repartitioning agents such as (but not limited to) beta agonists; Other coccidiostats such as (but not limited to) amprolium; Ionophores such as (but not limited to) lasalocid; Hops; Choline containing products; Tannins; Algae and/or sea-weed; Supplemental specific amino acids (ruminal protected or not rum inal protected); Rum inal protected long chain fatty acids; Non-microbial sourced enzyme concoctions; Short chain volatile fatty acids such as (but not limited to) butyric acid; Bambermycins; Antibodies produced from animal origin or plant origin that may or may not require going thru FDA approval. Examples include (but not limited to) blood serum, egg albumin, plant proteins, etc.; A variety of antibiotic feed additives; or a combination of the above.

The instant invention also includes a method for parasitic control, methane control, ammonia control, liver abscess control and/or nutritional supplementation in livestock by including the compound(s) described above in their diet.

Looking now to how the various results will be assessed in the instant application, there are multiple criteria which will be assessed. We will consider it a success if any one or more or any combination of the criteria are achieved. As follows:
a. If cattle fed Deccox/Yucca combination have similar or improved daily gain compared to cattle fed Rumensin/Tylan
b. If cattle fed Deccox/Yucca combination have similar or improved feed efficiency compared to cattle fed Rumensin/Tylan
c. If cattle fed Deccox/Yucca combination have similar or lower incidence of total liver abscesses compared to cattle fed Rumensin/Tylan
d. If cattle fed Deccox/Yucca have a similar or a lower incidence of severe liver abscesses compared to cattle fed Rumensin/Tylan
e. If cattle fed Deccox/Yucca combination have improved daily gain compared to cattle fed no feed additives
f. If cattle fed Deccox/Yucca combination have improved feed efficiency compared to cattle fed no feed additives
g. If cattle fed Deccox/Yucca combination have a lower incidence of total liver abscesses compared to cattle fed no feed additives
h. If cattle fed Deccox/Yucca have a lower incidence of severe liver abscesses compared to cattle fed no feed additives

### EXAMPLES

### Example 1

Feeding trials were completed with a randomized block design utilizing 4 treatments and 13 replicates per treatment. The first 20 pens (block 1, 5 replicates per treatment group) were placed first, the second 20 pens (block 2, 5 replicates per treatment group) were placed third, and the last 12 pens (block 3, 3 replicates per treatment group) were placed last. All cattle were housed within one area of the feedyard. Groups of cattle were systematically allocated to pens in the specific feeding area so that the same treatment groups did not occupy the same pens between blocks.

### Cattle Population

Crossbred steers (n=4,849) with an average weight of 590 kg+/−19.7 kg of various origins from the High Plains and Midwest were selected from within a northwest Kansas commercial feedyard. All cattle underwent the same receiving protocol and were placed in 200 head pens. All cattle were acclimated to feedlot rations and had been on feed a minimum of 60 days prior to trial enrollment. Starting rations during the initial 60 days included 75 mg/head/day tylosin (Tylan, Elanco Animal Health, Greenfield, Ind.) with 15 g/ton monensin (Rumensin, Elanco Animal Health, Greenfield, Ind.).

At 60 days of feed, 2-200 head pens were systematically allocated to 4 pens of 100 head. Two pens of 200 head were sorted at the chute into 4-100 head pens at time of terminal implant. Each 200 head pen contributed 50 head to each of the 4 pens that would be eventually allocated to treatment. This was accomplished by systematic allocation of 5 head at a time to each of the trial pens. If 200 head counts were not exactly 100 head/pen, the final allocations were that the number of animals per treatment group were similar. Once the 4-100 head pens (1 replicate) were sorted, the pens of cattle were weighed by pen, a 4% pencil shrink was applied, and pens were randomly assigned to 1 of 4 treatments: (1) Positive control (MT) 75 mg/head/day tylosin (Tylan,® Elanco Animal Health, Greenfield, Ind.) and 185 mg/head/day monensin (Rumensin,® Elanco Animal Health, Greenfield, Ind.); (2) Negative control (NO MT) fed without Rumensin® or Tylan®; (3) Decoquinate (DECO) fed 275 to 325 mg/head/day (Deccox,® Zoetis, Parsippany, N.J.); or (4) 3 g/head/day Yucca schidigera (YS) (Micro-Aid,® DPI Global, Porterville, Calif.). All cattle received an identification tag specific to the trial. Caretakers and processing staff were blinded to treatment.

### Diet Preparation

All treatment groups received the same base ration (Table 1) that was balanced to provide 14% crude protein DM, 2.25MJ/kg NEm, and 1.55 MJ/kg NE_{g} (Table 1). Rations were prepared using a stationary batcher box (Feeding Systems, Inc., Columbus, Nebr.) and loaded onto mixer/delivery trucks with box-mounted scale (Rotomix Manufacturing, Dodge City, Kans.). Treatment rations were delivered twice daily (0800 and 1300) on separate trucks to prevent cross contamination of treatment rations.

TABLE 1
Diet composition of diets containing tylosin phosphate
and monensin, no tylosin phosphate or monensin, decoquinate
alone, or Yucca schidigera extract alone.

| Item | |
|---|---|
| Ingredient (% As Fed) | |
| Corn oil | 1.2 |
| High moisture corn | 32.0 |
| Liquid supplement | 4.20 |
| Triticale silage | 12.50 |
| Steam-flaked corn | 34.10 |
| Wet distillers grains | 16.0 |
| Nutrient Composition (DM basis) | |
| CP, % | 13.94 |
| Roughage, % | 5.92 |
| NEₘ, MJ/kg | 2.25 |
| NE_{g}, MJ/kg | 1.55 |
| Salt, % | 0.31 |
| Calcium, % | 0.63 |
| Phosphorous, % | 0.40 |
| Magnesium, % | 0.31 |
| Potassium, % | 0.97 |
| Sulfur, % | 0.23 |

Freshly voided fecal samples (n=10 per pen) were collected at 50 d of feed for fecal flotation to determine the presence of Eimeria spss. Fecal samples were collected from 10 freshly voided fecal pats on the pen floor. Samples were collected in plastic bags and shipped on ice to a diagnostic lab within 48 hours of collection. Qualitative fecal floatation was performed using the Modified Wisconsin Technique double centrifugation fecal flotation using sheather's sugar solution as described by Zejac & Conboy (2012). Qualitative fecal flotation was selected due to cost and for greater diagnostic detection of the presence of Eimeria spss (<50 oocysts/gram). Qualitative fecal flotation measurements were measured on a scale of 1+ to 5+ with 1+ having <10 oocysts per slide, 3+ samples having 50-100 oocysts per slide, and 5+ having >500 oocysts/slide. This technique is considered semi-quantitative, with 3+ samples equivalent to approximately 50 oocysts/gram. An additional 2 fecal samples per pen were obtained (12 total/pen) in the previously described manner and composited into one sample pen for total tract digestibility analysis. These samples were stored frozen until analysis at a commercial laboratory.

### Performance Data

Data on cattle performance were derived from feedlot closeout reports after all pens of cattle were shipped to an abattoir. Daily dry matter intakes were recorded via bunk management personnel and entered into the computerized bunk management system. The average dry matter intake per pen over the 60-day period was retrieved from closeout data. Final body weight was calculated as (carcass weight/0.64). Average daily gain (ADG) was calculated on a pen basis using the equation ((Final body weight−Initial body weight)/days on feed). Feed conversion was calculated by dividing the total weight gained divided by the average daily dry matter intake, then carcass adjusted G:F was calculated as the reciprocal of feed conversion.

### Liver Abscess & Carcass Data

At 60±4 days of feed, all pens of cattle were shipped to the same abattoir. Hot carcass weight was collected from the plant via scale on carcass rail. Liver abscess scores were assessed by USDA trained professionals and data were collected by third party trained professionals using the liver scoring system previously described by Brown et al. (1975): 0=no abscesses, A−=2-4 abscesses under 1 inch in diameter, A=1-2 abscesses greater than 1 inch in diameter and A+=abscesses greater than 2 inches in diameter, ruptured abscess, or adhesions to body wall. Yield grade and quality grade data were collected from the abattoir by a third party.

### Statistical Analysis

Continuous variables were analyzed using PROC Glimmix (SAS 9.4, SAS Inst. Inc., Cary, N.C.). Models contained treatment as a fixed effect, block as a random effect, and pen as the experimental unit. Least squares means were compared using the PDIFF function of SAS and the Tukey-Kramer adjustment for multiple comparisons. Statistical significance was determined at P<0.05 with tendencies at 0.06>P<0.10. Liver abscess scores, yield grade, and incidence of Eimeria spss. were converted to pen level frequency percentages prior to analysis.

### Foote Cattle 60 Day Study

TABLE 2
Cattle Performance

| Item | MT | NO MT | DECO | YS | SEM | P-value |
|---|---|---|---|---|---|---|
| Initial | 593.4 | 589.9 | 593.5 | 593.6 | 9.5 | 0.04 |
| BW, kg | | | | | | |
| Carcass | 684.0^{a} | 675.4^{b} | 681.0^{a, b} | 683.4^{a} | 7.2 | 0.002 |
| adjusted | | | | | | |
| final | | | | | | |
| BW, kg | | | | | | |
| Carcass | 1.49^{a, b} | 1.44^{a} | 1.47^{a, b} | 1.53^{b} | 0.10 | 0.01 |
| adjusted | | | | | | |
| ADG, kg | | | | | | |
| DMI, kg | 12.1^{a, b} | 11.9^{a} | 12.1^{a, b} | 12.2^{b} | 0.55 | 0.06 |
| Carcass | 0.126 | 0.120 | 0.120 | 0.123 | 0.01 | 0.001 |
| adjusted | | | | | | |
| G:F, kg/kg | | | | | | |

### Cattle Performance

Cattle in the MT or YS groups had the heaviest final body weight while cattle in the DECO group were intermediate and cattle in the NO MT group where the lightest. (table 3). Compared to cattle fed MT, cattle fed NO MT had lower final body weight, lower ADG, consumed less food and were less efficient. Cattle in the DECO and YS groups exhibited growth performance more similar to cattle in the MT group.

### Carcass Characteristics

HCW mirrored that of final weight (Table 3) with the heaviest being the cattle in the MT and YS groups, the lightest being in the NO MT group, and the DECO group being in the intermediate. Carcass performance between all treatment groups were consistent, with >70% of all treatment groups within yield grades 2 & 3 and >80% of all cattle grading USDA choice.

TABLE 3
Carcass Performance

| Item | MT | NO MT | DECO | YS | SEM | P-value |
|---|---|---|---|---|---|---|
| HCW, kg | 437.8^{a} | 432.3^{b} | 435.8^{a, b} | 437.4^{a} | 3.1 | 0.002 |
| Dressing yield, % | 64.1^{a} | 63.9^{a, b} | 63.9^{a, b} | 63.8^{b} | 0.5 | 0.02 |
| YG1, % | 7.6^{a} | 10.8^{b} | 7.0^{a, c} | 8.0^{a, b, c} | 1.4 | 0.01 |
| YG2, % | 32.8 | 30.1 | 33.8 | 32.8 | 2.7 | 0.47 |
| YG3, % | 40.6 | 42.5 | 40.8 | 40.7 | 2.6 | 0.81 |
| YG4, % | 16.8 | 15.0 | 17.1 | 17.0 | 2.1 | 0.67 |
| YG5, % | 2.1 | 1.1 | 1.3 | 1.4 | 0.6 | 0.40 |
| Prime, % | 2.2 | 1.5 | 2.4 | 1.9 | 0.4 | 0.39 |
| Choice, % | 81.1 | 80.3 | 83.5 | 81.6 | 2.2 | 0.54 |
| Select, % | 15.1 | 16.4 | 11.9 | 14.7 | 1.9 | 0.11 |
| Standard, % | 1.3 | 1.7 | 2.1 | 1.5 | 0.6 | 0.78 |

### Liver Scores & Eimeria

Pairwise comparisons of least squares means found significant differences. Cattle supplemented with monensin and tylosin compared to cattle without monensin and tylosin had less A+ liver abscesses and less total liver abscesses. For cattle in the DECO or YS groups, the incidence of liver abscesses was higher than cattle in the MT group, but lower than cattle in the NO MT group, but not statistically significant from either.

TABLE 4
Liver Abscesses & Coccidiosis

| Item | MT | NO MT | DECO | YS | SEM | P-value |
|---|---|---|---|---|---|---|
| A+, % | 16.25^{a} | 23.15^{b} | 19.68^{a, b} | 19.02^{a, b} | 2.1 | 0.02 |
| A, % | 3.8 | 5.3 | 4.1 | 4.6 | 0.95 | 0.33 |
| A−, % | 7.7 | 7.0 | 7.6 | 7.5 | 1.5 | 0.84 |
| Total liver | 27.7^{a} | 35.5^{b} | 31.4^{a, b} | 33.2^{a, b} | 3.6 | 0.06 |
| abscess, % | | | | | | |
| Eimeria spp. | 0.0 | 3.0 | 5.0 | 0.0 | 1.9 | 0.18 |
| incidence, % | | | | | | |

These results are consistent with several studies that indicate tylosin administration reduced the number of A+ liver abscesses found at slaughter. It appears that the change in microbiome occurs as soon as the antimicrobial is removed and continues over time.

There were no treatment effects for the difference in Eimeria spss incidence between treatment groups. The highest total incidence was seen in the decoquinate treatment group, with 5.0% of samples positive for at least 1+Eimeria (Table 4). Within pen samples, incidence was as high as 30% positive samples for Eimeria (data not shown). However, none of the positive samples achieved any more than 3+ on the qualitative scale, which if converted to quantitative fecal counts, would be equivalent to 50 oocysts/gram (KSU VDL, personal communication). This amount is not considered pathogenic, as coccidiosis clinical signs occur with infections as high as 100,000 oocysts/gram. There were no differences in the measured nutrient content in fecal samples. (Table 5).

TABLE 5
Fecal Analysis

| Item | MT | NO MT | DECO | YS | SEM | P-Value |
|---|---|---|---|---|---|---|
| CP % DM | 18.4 | 19.1 | 19.0 | 19.6 | 0.75 | 0.16 |
| ADF % DM | 34.5 | 31.3 | 31.7 | 30.6 | 02.3 | 0.14 |
| Fat % DM | 2.23 | 2.46 | 2.43 | 2.38 | 0.12 | 0.43 |
| Starch % DM | 3.34 | 4.27 | 4.22 | 3.49 | 1.2 | 0.28 |

### Example 2

### Foote Cattle 120 Day Study

Feeding trials were completed with a randomized complete block design utilizing 3 treatments and 18 replicates per treatment. Blocking factor in this study was each group of three pen reps. All cattle were housed within one area of the feedyard.

### Cattle Population

Crossbred steers (n=6804) with an average weight of 387 kg of various origins from the High Plains and Midwest were selected from within a northwest Kansas commercial feedyard. All cattle underwent the same receiving protocol and were allotted to pen 5 head at a time. Head counts varied from 100 hd/pen to 150 hd/pen depending on pen size. Once the group of three pens were sorted (1 replicate), the pens of cattle were weighed by pen, a 4% pencil shrink was applied, and pens were randomly assigned to 1 of 3 treatments: (1) Positive control (MT) 75 mg/hd/day tylosin (Tylan,® Elanco Animal Health, Greenfield, Ind.) along with 350 mg/hd/d of monensin (Rumensin,® Elanco Animal Health, Greenfield, Ind.); (2) Negative control (NO MT), and (3) (DY) Decoquinate fed at 300 mg/hd/day (Deccox,® Zoetis, Parsippany, N.J.) and Yucca Schidigera fed at 3 g/hd/d (Micro-Aid,® DPI Global, Porterville, Calif.). All cattle were transitioned to the final diet over a 25-day period using a common set of transition diets. Once cattle were on the final diets, experimental diets were fed for the remainder of the study (120-130 days). All cattle received an identification tag specific to the trial. Caretakers and processing staff were blinded to treatment.

### Diet Preparation

All treatment groups received the same base ration (Table 6). Rations were prepared using mixer trucks with box-mounted scale (Rotomix, Dodgy City Kans.). Treatment rations were delivered twice daily (0800 and 1300) on separate trucks to prevent cross contamination of treatment rations.

TABLE 6
Diet composition of diets containing tylosin phosphate
and monensin, no tylosin phosphate or monensin, decoquinate
alone, or Yucca shidigera extract alone.

| Item | |
|---|---|
| Ingredient (% AF) | |
| Corn oil | 1.2 |
| High moisture corn | 16 |
| Liquid starter | 4.3 |
| Corn silage | 19 |
| Dry ground hay | 2 |
| Steam-flaked corn | 37.5 |
| Wet distillers grains | 20 |
| Nutrient Composition (DM basis) | |
| CP, % | 12.94 |
| Roughage, % | 8.41 |
| NEₘ, MJ/kg | 2.20 |
| NE_{g}, MJ/kg | 1.51 |
| Salt, % | 0.33 |
| Calcium, % | 0.69 |
| Phosphorous, % | 0.41 |
| Magnesium, % | 0.33 |
| Potassium, % | 1.00 |
| Sulfur, % | 0.23 |

### Performance Data

Data on cattle performance were derived from feedlot closeout reports after all pens of cattle were shipped to an abattoir. Daily dry matter intakes were recorded via bunk management personnel and entered into the computerized bunk management system (Animal Health International, Greely, Colo.). The average dry matter intake per pen was retrieved from closeout data. Final body weight was calculated as (carcass weight/0.64). ADG was calculated on a pen basis using the equation ((Final body weight−Initial body weight)/days on feed). Feed conversion was calculated by dividing the total weight gained divided by the average daily dry matter intake.

### Liver Abscess & Carcass Data

All pens of cattle were shipped to the same abattoir (National Beef, Liberal, Kans.). Hot carcass weight was collected from the plant via scale on carcass rail. Liver abscess scores were assessed by USDA trained professionals and data were collected by third party trained professionals (CattleTrail, Inc. Dodge City, Kans.) using the liver scoring system previously described by Brown et al. (1975): 0=no abscesses, A−=2-4 abscesses under 1 inch in diameter, A=1-2 abscesses greater than 1 inch in diameter and A+=abscesses greater than 2 inches in diameter, ruptured abscess, or adhesions to body wall. Yield grade and quality grade data were collected from the abattoir by a third party (CattleTrail, Inc. Dodge City, Kans.).

TABLE 7
Cattle Performance

| Item | NO MT | DY | MT | SEM | P-value |
|---|---|---|---|---|---|
| Initial BW, K_{g} | 387.2 | 387 | 387.7 | 7.0 | 0.77 |
| Carcass adjusted | 679.2 | 679.2 | 677.5 | 8.1 | 0.67 |
| final BW, K_{g} | | | | | |
| Carcass adjusted | 1.89 | 1.88 | 1.87 | 0.05 | 0.31 |
| ADG, K_{g} | | | | | |
| DMI, K_{g} | 11.9^{a, b} | 12.0^{a} | 11.8^{b} | 0.2 | 0.05 |
| Carcass adjusted | 6.33 | 6.40 | 6.32 | 0.08 | 0.29 |
| F:G | | | | | |

Cattle growth performance was similar across treatments except for dry matter intake. Cattle in the DY group consumed more feed compared to cattle in the MT group (Table 7).

TABLE 8
Carcass Performance

| Item | NO MT | DY | MT | SEM | P-value |
|---|---|---|---|---|---|
| HCW, K_{g} | 434.7 | 434.7 | 433.6 | 5.2 | 0.67 |
| Dressing yield, % | 64.1^{a} | 63.9^{a, b} | 63.7^{b} | 0.12 | 0.02 |
| YG1, % | 8.3 | 7.2 | 7.4 | 1.8 | 0.51 |
| YG2, % | 27.0^{a} | 30.9^{b} | 30.0^{a, b} | 2.2 | 0.09 |
| YG3, % | 44.0 | 42.7 | 40.6 | 2.3 | 0.45 |
| YG4, % | 20.2 | 17.4 | 17.9 | 2.3 | 0.29 |
| YG5, % | 8.3 | 7.2 | 7.4 | 1.8 | 0.51 |
| Prime & choice, % | 87.1^{a} | 83.9^{b} | 84.6^{a, b} | 1.8 | 0.03 |
| Select, % | 11.6^{a} | 14.5^{b} | 13.3^{a, b} | 1.8 | 0.08 |
| Standard, % | 1.3^{a} | 1.6^{b} | 2.1^{c} | 0.7 | 0.08 |

Cattle in the NO MT group had a higher dressing percent compared to cattle in the MT group. Overall carcass quality was very good within industry standards (Table 8).

TABLE 9
Liver abscesses

| Item | NO MT | DY | MT | SEM | P-value |
|---|---|---|---|---|---|
| A+, % | 22.5^{a, b} | 18.1^{b, c} | 13.3^{c} | 1.9 | 0.0068 |
| A, % | 2.9 | 2.5 | 2.2 | 0.4 | 0.37 |
| A−, % | 7.2^{a, b} | 8.7^{a} | 6.8^{b} | 0.8 | 0.08 |
| Total liver abscess, % | 32.7^{a} | 29.4^{a, b} | 22.4^{b} | 2.3 | 0.012 |

Consistent with the initial study, there were significant differences in liver abscesses for both A+ livers and total liver abscesses (Table 9). Cattle in the MT group had significantly less liver abscesses compared to cattle in the NO MT group and abscess scores were intermediate in cattle in the DY group.

Based on the results of this study, there is the potential to eliminate the use of feed grade antimicrobials within the last 120, 90, or 60 days of the feeding period, with minimal effects on cattle performance, carcass quality, liver abscess incidence, or coccidiosis prevention. It is clear that antimicrobial alternatives such as decoquinate or Yucca schidigera extract have a positive effect on cattle performance, feed efficiency, or carcass quality.

Any method described herein may incorporate any design element contained within this application and any other document/application incorporated by reference herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present invention may be embodied in other forms without departing from the spirit and the essential attributes thereof, and, accordingly, reference should be made to the appended claims, rather than to the foregoing specification, as indicating the scope of the invention. The invention illustratively disclosed herein suitably may be practiced in the absence of any element which is not specifically disclosed herein.

1. A food supplement composition for a non-human animal comprising decoquinate and sarsaponin.

2. The food supplement composition of claim 1 wherein the components are in liquid form, solid form, or a combination thereof.

3. The food supplement composition of claim 1 which contains decoquinate in an amount which delivers 0.1 to 10.0 mg/kg body weight (BW) and sarsaponin in an amount which delivers 50 to 1000 mg/kg feed BW.

4. The food supplement composition of claim 1, further comprising at least one grain by-product.

5. The food supplement composition of claim 1, wherein the grain by-product is selected from the group comprising oats, barley, maize, lupins, lupin hulls, bran, canola meal, soya meal and combinations thereof.

6. The food supplement composition of claim 1, further comprising hay and silage.

7. The food supplement composition of claim 5, wherein the hay is at least one of oaten, wheaten, silage, and meadow hay.

8. The food supplement composition of claim 1, further comprising at least one pharmaceutical composition.

9. The food supplement composition of claim 1, further comprising at least one nutrient.

10. The food supplement composition of claim 7, wherein the at least one nutrient is at least one of a vitamin and a mineral.

11. The food supplement composition of claim 1, wherein the desired physical form is a pellet, a crumble, a mash, or a lick.

12. The food supplement composition of claim 1, further comprising a flavoring.

13. The food supplement composition of claim 1, wherein the flavoring includes molasses.

14. The food supplement composition of claim 1, further comprising a pharmaceutical composition comprising an animal food supplement composition as an active ingredient optionally admixed with a pharmaceutically acceptable carrier.

15. The food supplement composition of claim 1, further comprising one or more strains of bacteria.

16. The food supplement composition of claim 1, further comprising one or more essential oils.

17. The food supplement composition of claim 1, further comprising any forage based plant that can be fed to cattle as either hay or silage including alfalfa, sorghum/sudan, whole plant corn, whole plant soybeans, whole plant peas, and the like.

18. A method for parasitic control, methane control, ammonia control, liver abscess control and nutritional supplementation in livestock by including the compound from claim 1 in their diet.
